(19) United States
(12) Patent Application Publication
(10) Pub. No.: US 2026/0038670 A1
(43) Pub. Date: Feb. 5, 2026

(54) A COMPUTER-IMPLEMENTED METHOD OF DETERMINING IF A FUNDUS IMAGE REQUIRES REFERRAL FOR INVESTIGATION FOR A DISEASE

(71) Applicant: AI SIGHT LIMITED, Daresbury, Cheshire (GB)

(72) Inventors: Yalin ZHENG, Liverpool, Merseyside (GB); David WONG, Liverpool, Merseyside (GB); Mark JOHNSON, Liverpool, Merseyside (GB); Simon HARDING, Liverpool, Merseyside (GB); Gabriela CZANNER, Liverpool, Merseyside (GB); Dongxu GAO, Liverpool, Merseyside (GB); Wenyue ZHU, Liverpool, Merseyside (GB)

(21) Appl. No.: 18/997,364

(22) PCT Filed: Jul. 6, 2023
(86) PCT No.: PCT/GB2023/051792

§ 371 Date: Jan. 21, 2025

(30) Foreign Application Priority Data
Jul. 20, 2022 (GB) 2210623.1

(51) Int. Cl. G16H 30/40

(52) U.S. Cl. CPC G16H 30/40

(57) ABSTRACT

The present disclosure relates to a computer-implemented method of determining if a fundus image requires referral for investigation for a disease. The method comprises: performing a pairwise comparison of the fundus image against each example fundus image in a reference image set using one or more machine learning algorithms to determine a difference in severity of the fundus image compared to each example fundus image in the reference image set; and flagging the fundus image as requiring referral for investigation for the disease based on results of the pairwise comparisons, wherein the reference image set includes a plurality of example fundus images, the plurality of example fundus images ranked according to their degree of severity of disease.

### FIELD

The subject-matter of the present disclosure relates to determining if a fundus image requires referral for investigation of a disease and ranking a plurality of images within a reference image set. Specifically, but not exclusively, the disease may be an ocular disease, for example, diabetic retinopathy.

### BACKGROUND

Ocular diseases, such as diabetic retinopathy, can be diagnosed by observing fundus images. Typically, a diagnosis is made by a trained medical professional. In certain areas, e.g. those with high population density, there may be insufficient medical personnel who are trained to assess the high number of fundus images from patients suspected as having an ocular disease such as diabetic retinopathy.

Artificial intelligence (AI) methods have been proposed to categorise fundus images as having disease and not having disease. However, such methods have insufficient accuracy and flexibility.

It is an aim of the present subject-matter to alleviate such problems and improve on prior art methods.

### SUMMARY

According to an aspect of the present disclosure, there is provided a computer-implemented method of determining if a fundus image requires referral for investigation for a disease, the method comprising: performing a pairwise comparison of the fundus image against each example fundus image in a reference image set using one or more machine learning algorithms to determine a difference in severity of the fundus image compared to each example fundus image in the reference image set; and flagging the fundus image as requiring referral for investigation for the disease based on results of the pairwise comparisons, wherein the reference image set includes a plurality of example fundus images, the plurality of example fundus images ranked according to their degree of severity of disease.

A machine learning algorithm is able to decide whether one image is more severe than another image with a higher degree of accuracy than trying to classify how severe a disease state is in an image directly.

The computer-implemented method may further comprise: determining a position of the fundus image against the example fundus images within the reference image set; and comparing the position of the fundus image with a threshold for referral, wherein the flagging the fundus image as requiring referral comprises flagging the fundus image as requiring referral if the position of the fundus image is above the threshold for referral. In this way, the referral threshold can be set to adjust the sensitivity depending on local referral threshold in different countries/regions.

The one or more machine learning algorithms may comprise one or more neural networks.

The or each neural network may be a convolutional neural network

The one or more convolutional neural networks may be a plurality of convolutional neural networks.

Each of the plurality of convolutional neural networks may be trained on a different data set. Training each network on a different data set ensures independence between networks.

The computer-implemented method may further comprise amalgamating the results of the pairwise comparisons from each convolutional neural network, and wherein the sending the fundus image for referral for investigation for the disease may be based on the amalgamated pairwise comparisons. Using a plurality of convolutional neural networks and amalgamating the results means that smaller data sets can be used.

The amalgamating of the results of the pairwise comparisons may comprise supplying the results of the pairwise comparison as inputs to one or more lasso regression models, the or each lasso regression model having a decision boundary associated with a threshold of disease severity.

The or each lasso regression model may be a plurality of lasso regression models and wherein the threshold of disease severity for each model may be different. In this way, it is possible to categorise the disease state in terms of its severity.

The computer-implemented method may further comprise estimating a certainty value of a probability of needing referral. The certainty value may also be called a certainty or uncertainty interval. The certainty value takes account of complexity in images.

The estimating may comprise performing bootstrapping.

The amalgamating of the results of the pairwise comparison may comprise accumulating results from each convolutional neural network, and comparing the accumulated results to the threshold for referral. In such a method, no training of a model such as a Lasso regression model is required.

The amalgamating the results comprises setting a plurality of thresholds of disease severity within the reference image set, and determining a frequency of occurrence of the fundus image above each of the thresholds of disease severity.

The threshold for referral may correspond to one of the plurality of thresholds of disease severity.

The amalgamating the results of the pairwise comparison may comprise fitting an s-curve to the results of each convolutional neural network; determining a probability of requiring referral based each fitted s-curve; and performing linear discriminant analysis on the determined probabilities.

The computer-implemented method may further comprise selecting a subset of the plurality of convolutional neural networks using a selecting algorithm.

The selecting algorithm may comprise a lasso regression model, the lasso regression model having a decision boundary associated with the threshold for referral, the selecting may comprise applying the results from each convolutional neural network into the lasso regression model, ordering the convolutional neural networks in terms of accuracy at predicting referral, and selecting a predetermined number of the highest ranked convolutional neural networks as the subset. In this way, computation time is reduced.

The computer implemented method of any preceding claim may further comprise: receiving the reference image set; and performing pairwise comparison of each image of the plurality of images within the reference image set against every other image of the plurality of images within the image set; and ranking the plurality of example fundus images according to a degree of severity of disease based on the pairwise comparisons.

The disease may be diabetic retinopathy.

According to another aspect of the present disclosure, there is provided a computer-implemented method of ranking a plurality of images within a reference image set, the method comprising: receiving the reference image set; and performing pairwise comparison of each image of the plurality of images within the reference image set against every other image of the plurality of images within the image set; and ranking the plurality of example fundus images according to a degree of severity of disease based on the pairwise comparisons.

The disease may be diabetic retinopathy.

According to another aspect of the present disclosure, there is provided a method of training a machine learning model to perform pairwise comparison using a reference data set having a plurality of references images ranked according to their degree of severity of disease, the method comprising: providing the reference data set; providing a training data set of images having different severity of disease, each image in the training data set having a label indicating where the image fits within the reference images of the reference data set; and training the machine learning algorithm to determine a location within the reference data set of each image in the training data set.

A transitory or non-transitory computer-readable medium including instructions stored thereon that when executed by one or more processors cause the processor to perform any of the methods described above.

### BRIEF DESCRIPTION OF DRAWINGS

The subject-matter of the present disclosure is best understood with reference to the accompanying figures, in which:

FIG. 1 shows a detailed flow chart of a computer-implemented method of categorising a fundus image as requiring further investigation for disease, according to an embodiment;

FIG. 2 shows tabulated details of training sets used to train a neural network used in the method from FIG. 1;

FIG. 3 shows a result of using the method from FIG. 1 to categorise a fundus image as requiring further investigation for disease, according to an embodiment;

FIG. 4 shows a high-level flow diagram of pairwise comparison performed according to an embodiment;

FIG. 5 shows a schematic of a convolutional neural network used for performing pairwise comparison according to an embodiment; and

FIG. 6 shows a flow chart of ranking fundus images within a reference image set according to an embodiment.

### DESCRIPTION OF EMBODIMENTS

All methods described herein may be computer-implemented methods, where each step is implemented on a computer. The computer may include a data store (or data storage), and one or more processors. The computer-implemented methods may be provided as instructions on a transitory, or non-transitory, computer-readable medium. The transitory or non-transitory computer-readable medium may be provided on the data store. The instructions, when executed by the one or more processors, configure the processor to perform the method steps described herein.

It will be appreciated that, whilst the following description describes one or more embodiments, features from different embodiments may be used in other embodiments without introducing new subject-matter that extends beyond the content of the present disclosure.

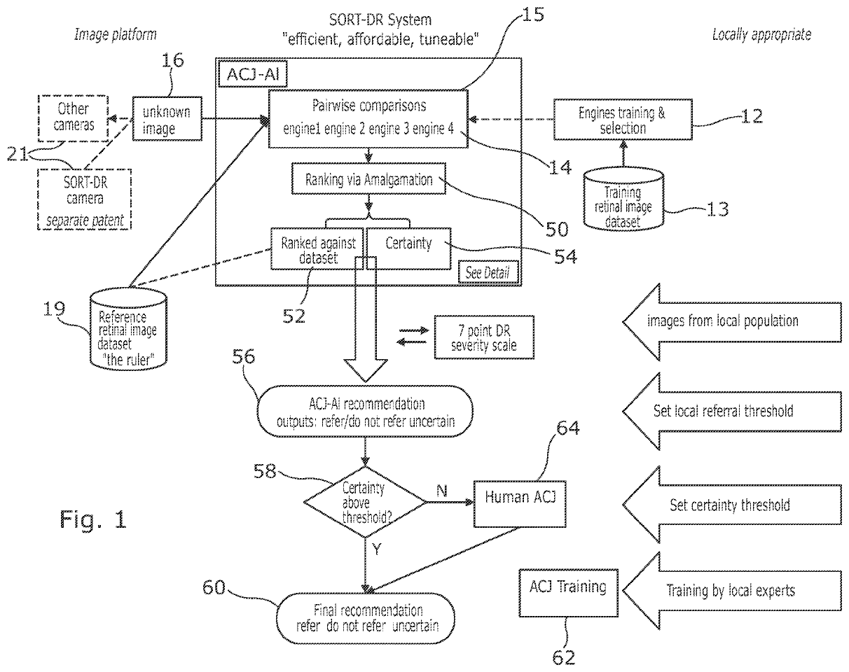

With reference to FIG. 1, the method starts at step 12 by training one or more neural networks using training image sets stored in a database 13. In some embodiments, there are a plurality of neural networks, for example 25 neural networks. The neural networks may be convolutional neural networks.

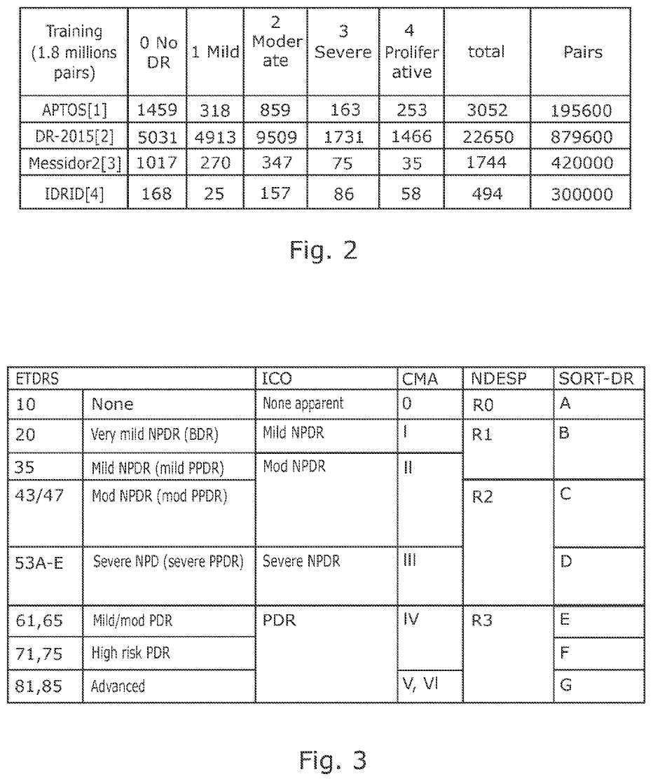

A plurality of training sets were used to train each of the neural networks. A different training set may be used to train each neural network. The different training sets have been created from a pool of training image sets. The pool of training image sets may include (Asia Pacific TeleOphthalmology Society) APTOS 2019 Blindness Detection Dataset (n=5,590), Kaggle-EyePACS (n=88,702), Methods to Evaluate Segmentation and Indexing Techniques in the field of Retinal Ophthalmology (in French) Messidor-2 2 (n=1,744), and the Indian Diabetic Retinopathy Image Dataset (IDRiD) (n=494), where n is a number of fundus images within the training image set. The pool of training image sets referred to herein are publicly available and are shown in FIG. 2 how the details of the subset we used from each of the above (e.g. total number of images used from each of them, the number of images corresponding different severities), and the total number of pairs of imaged we have generated.

It should be noted that disease severity for diabetic retinopathy (DR) is graded according to different levels of severity. Different medical authorities have different ways to grade severity level. As shown in FIG. 3, there are many medical authorities including International Council of Ophthalmology (ICO), Chinese Medical Association (CMA), and National Health Service Diabetic Eye Screening Programme (NDESP). Each of those has different categories describing severity of diseases.

Referring back to FIG. 2, the ICO standard of disease severity has been used to categorise the four data sets. The categories of severity include 0 no diabetic retinopathy (DR), 1 mild DR, 2 moderate DR, 3 severe DR, and 4 proliferative DR. There are five categories in total.

A plurality of training sets is generated from the four data sets. There may be a one-to-one mapping of a training set to a neural network. In this case, there may be 25 training sets and 25 neural networks. The 25 training sets may be constructed by taking samples from each of the four image sets described above. Whilst the number from each of the image sets does not matter, there are images for each of the five categories within each of the 25 training sets. Augmentation may be used to increase the number of training examples within each training set. For example, augmentation such as flip and rotations of the images may be performed. One advantage of using pairwise comparison is to obtain accurate comparison classification with fewer images. The number of pairs is in a proportion of n(n-1) where n is the number of images, which will be exponentially larger than the number of images. For example, a further subset of 28,000 (precisely 27940) images (the subset of the four public data sets described above and in FIG. 2) derives ˜1.8 million pairs.

Each neural network is trained using its designated training set. By using different training sets, independence of each trained neural network is ensured.

The validation data set may be the (Diabetic Retinopathy Dataset) DDR data set from China (n=13,673).

With further reference to FIG. 1, the neural networks (AI) 14 are trained to perform pair wise comparisons of two images at step 15. One of those images 16 may be an image from a reference set stored in a reference database 19, and the other may be a patient's image. The patient's image may be captured using one or more cameras 21, for example. The one or more cameras 21 in FIG. 1 may be replaced with any camera platform suitable for the purposes of obtaining fundus images, preferably colour fundus images.

The patient's fundus image(s) are pre-processed to be 512×512 pixels. Other sizes are also envisaged, and this particular size is given for illustrative purposes only. The example fundus images of the ruler may also be pre-processed to be of the same size, or at least similar.

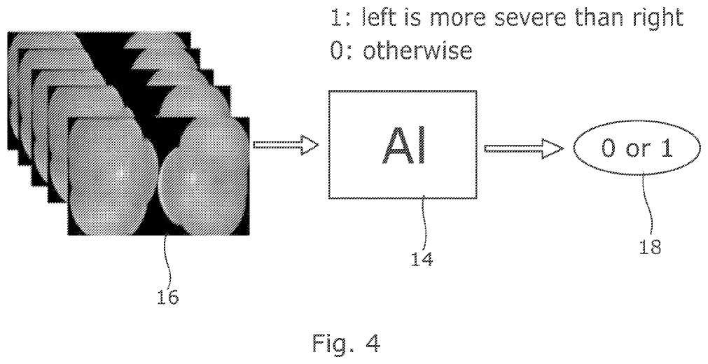

With reference to FIG. 4, the images may be colour fundus images 16. An output from the neural networks 14 may be a decision 18 as to whether a first image 16 has more severe DR than a second image 16. The terms first and second may be used interchangeably with the terms left and right, because the images being compared may be positioned side by side. The result may be binary, e.g. 0 or 1, where 1 signifies that the first image has more severe DR than the second image, or vice, versa.

More specifically, with reference to FIG. 5, a pair of fundus images A and B may be compared using the neural network 14. Each image 16 may be supplied to an encoder 20 for feature extraction. The image 16 may be supplied as an input vector of grayscale values in RGB format, for example. The grayscale values may be concatenated together to form the input vector. A series of encoding operations are performed on each image in parallel. The encoding operations include convolution performed by convolution layers. The encoding operations also include pooling and dropout layers. In this way, the encoder 20 is able to learn features from each of the fundus images 16. The features are constructed as a feature vector 22. There are two feature vectors, A and B, one for each input image being compared. A summing layer 24 is included to sum together the feature vectors 22. The summing layer 24 may perform the addition in various ways, including subtracting the individual integers within the feature vectors 22, taking an average value of corresponding integers from the feature vectors, or concatenating the feature vectors together 22. The output from the summing layer 24 is a merged feature vector 26.

The merged feature vector 26 is applied to one or more fully connected layers to obtain a binary result, e.g. 1 or 0, where 1 signifies that the first image has more severe disease than the second image, or vice versa depending on the convention used. The result may also be a probability, e.g. between 0 and 1, as an alternative to being a binary result, e.g. 0 or 1.

The convolutional neural networks of the embodiments herein are different from the prior art at least in that they compare severities between a pair of images (i.e. differences), whereas prior art models such as Siamese and SimCLR strategies learn features via comparing similarities between two images.

It will be appreciated that a binary classification is useful for understanding a comparative severity of disease compared to a reference image. A referral for further investigation is achieved when the reference image is at a threshold for referral. Whilst this has some merit, such thresholds can and often need to change when new medical knowledge is obtained, and depending on referral culture within a local region, and even budgetary constraints where patients may need to wait for more severe disease before being referred. To address this issue, a reference image set 19 is employed which contains a plurality of example fundus images ranked according to their severity of disease, pairwise comparison is performed using the ranked images to slot the patient's image within the ranked list, and a threshold for referral may be applied to the results, as described in more detail below.

The reference image set 19 referred to herein is called a ruler. The ruler may include a plurality of images. For example, the ruler may include 158 images. This number may change according to specific embodiments but is found to be suitable for the purposes of diabetic retinopathy. For the purposes of diabetic retinopathy, we may refer to the ruler also as SORT-DR. With reference to FIG. 3, the images within the ruler include example fundus images within each class of severity. The number of example fundus images in each class is selected to be approximately the same, or even the same. The number of images per class is taken to be the number of images from the class which has the least number of example fundus images. The ruler includes its own classes, having more classes than the other standard class systems. There are seven classes within SORT-DR. Those classes are “None”, “Very mild NPDR”, “Mild NPDR”, “Moderate NPDR”, “Severe NPNR”, “mild/moderate PDR”, “High risk PDR”, and “advanced”. The acronym PDR is proliferative diabetic retinopathy. The acronym NPDR is non-proliferative diabetic retinopathy. The SORT-DR image set includes images within each of its seven classes.

To obtain the ranking, pairwise comparison is performed using the neural networks to compare each image against every other image in the reference set 19. The images can be ranked depending on how many images are more/less severe than it, e.g. counting the 1s and 0s. In this way, ranking is performed based on the pairwise comparisons.

Referring again to FIG. 1, at step 15, pairwise comparison of the patient's fundus image is performed on each image within the SORT-DR. This is performed using each neural network. In some embodiments, the number of neural networks can be reduced to a subset of neural networks. Reducing the number of neural networks to a subset is described in more detail below.

Once the patient's fundus image has been applied to each neural network, a set of results are obtained. When the ruler includes 158 reference images, the results will be 158 binary numbers (e.g. 0 or 1). For example, there may be one hundred 0s, where the patient's fundus image is less severe than the first one hundred example fundus images in SORT-DR. There may also be 58 1s where the patient's fundus image is more severe than the last 58 images within SORT-DR. Of course, some comparisons may be uncertain and there may not be a clear boundary between the sets of 1s and a clear set of 0s, e.g. there could be a middle of the output vector including both 0s and 1s mixed.

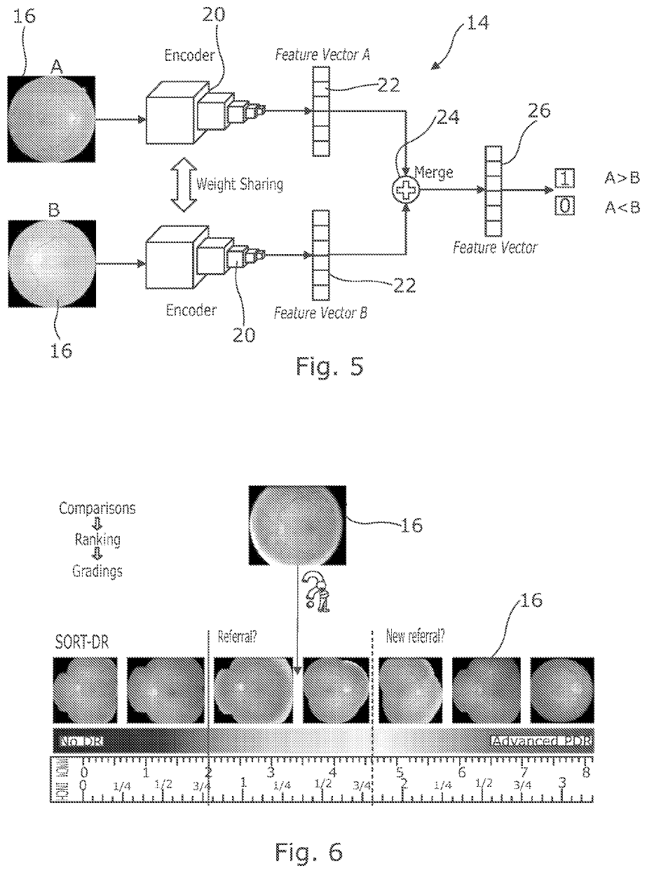

Once all neural networks (engines) 14 have provided an output vector, the data is ranked by amalgamation at step 50. At step 52, the result of the amalgamation is ranked against the ruler. In other words, a position within the ruler is obtained for the patient's fundus image. This can be seen more clearly with reference to FIG. 6, which shows how the patient's fundus image 16 is slotted within with ruler's example images 16. A certainty score is obtained at step 54 providing a degree of certainty regarding the position of the patient's fundus image within the ruler.

It is noted that by “slotting” the patient's fundus image within the ruler, the rank of the patient's image with respect to the images in the ruler is obtained. The ruler does not acquire the patient's fundus image as an additional example fundus image. In other words, the ruler will contain the same number of example fundus images before and after the comparison has been performed. However, in some embodiments, the ruler can be augmented offline by adding the patient's image if desired.

In one embodiment, the amalgamation is performed using a group Lasso regularised logistic regression model.

Using the group Lasso regularised logistic regression model, a plurality of thresholds of disease severity are set within the ruler. Each threshold of disease severity is located at a boundary between seven classes of the ruler, SORT-DR (see FIG. 3).

The Lasso regression model may be trained using cross validation using images from the ruler to select the optimal regularisation parameter. The cross validation may be 10-fold cross validation. The decision boundary of the group Lasso regularised logistic regression model may be one of the thresholds of disease severity. There may be a plurality of Lasso regression models trained, each having a different decision boundary associated with a different threshold of disease severity. In this way, different Lasso regression models may be used to adjust the severity of the referral threshold. In this way, the threshold of disease severity of the selected Lasso regression model may be considered a threshold for referral.

The output vectors from each of the 25 neural networks may be applied to the Lasso regression model. The best performing neural networks may be selected as a subset for a specific referral threshold. The number of engines within the subset may be decided using statistical measures including, for example cross validation error, prediction error, accuracy, sensitivity, specificity and area under the receiver operating characteristics curve. By reducing the number of neural networks to a subset, computation time may be reduced in the implementation without compromising the accuracy.

The output from the group Lasso regularised logistic regression model may be a binary outcome, for example 0 or 1, regarding whether the patient's fundus image should be sent for referral or not, and a predicted probability of the patient's fundus image should be sent for referral. The optimal discrimination rule is derived to separate referral images from non-referrals based on Youden J's statistics (J=sensitivity+specificity−1).

As described above, the output vectors from the neural networks may be used as input for the group Lasso regularised logistic regression model, and there may be some uncertainty regarding the vectors from the neural networks. At step 54, bootstrapping may be used to propagate such uncertainty and to estimate the credible interval for the output predicted probability from the group Lasso regularised logistic regression model. The width of the credible interval may be used to determine a certainty value. The certainty value may be communicated to the user as a %.

In another embodiment, instead of Lasso regression, the Bradley-Terry-Luce (BTL) model may be used. In this embodiment, no training is required to produce the ranking. Instead, the output vectors from the neural networks 14 (or the subset thereof), are tabulated using a comparison matrix. Each cell of the matrix is an accumulated score of corresponding values from each of the output vectors. The cells of the matrix are compared to a threshold for referral.

In another embodiment, a frequency approach may be used. In such an embodiment, pairwise comparison is performed on the plurality of neural networks, using the ruler image set, i.e., SORT-DR, and another known image set as the reference image set, e.g., ISDR dataset. The reference images include images with all and different disease severity, and their comparison results against the ruler images are known. The patient's fundus image is compared against all reference images, and a score can be calculated based on the frequency that the reference images are more severe than the patient's image. This score can be used to map to the ruler images, and to determine the rank of the patient's fundus image within the ruler images. A threshold for determining the severity of the patient's fundus image is applied based on the rank and severity of the ruler images, e.g. rank<=21, the patient's fundus image is a SORT-DR A; 21<rank<=79, it is a B or C; 79<rank<=102, it is a D; 102<rank<=159, it is a E or F or G. One severity class of the patient's fundus image can be determined by output vectors from one neural network. If output vectors from multiple neural networks are used, majority voting can be applied to determine the final severity class of the patient's fundus image.

In another embodiment, a S-curve may be used. This method can be divided into two steps.

The first step is the original S-curve fitting step, which is a regression-based method to produce an estimated score for each patient's image. For each patient's image, one AI pairwise comparison engine gives comparison results against 158 reference images, including 158 0s/1s with the corresponding probabilities for each pair of comparison. One S-curve is then fitted for each patient image to describe the relationship between 158 probabilities from one AI pairwise comparison engine and the normalised score of the ruler images. The score for the patient image is then estimated where the S-curve crosses the 0.5 horizontal line, which corresponds to a probability value of 0.5.

The second step is linear discriminant analysis. It utilises the estimated score from one or more S-curves and amalgamates the scores from multiple AI pairwise comparison engines to predict the disease severity for each patient image.

In another embodiment, the 7 classes of the reference dataset Sort-DR are used as predictors. The results of pairwise comparisons of each and every image from the DDR dataset were compared with each and every image of the Sort-DR dataset. The output of the pairwise comparisons is the probability that one image of each pair is more severe than the other. For example, the first image in the DDR data is compared with the 20 images of class A of the Sort-DR dataset. The mean of the 20 probabilities is calculated. Similarly, the means of this first DDR image is compared with each of the images of classes B, C, D, E, F, G.

Means of the probabilities from pairwise comparisons: The DDR database is divided into 2 parts based on a pre-determined threshold. For example, if we wished to distinguish between moderate and severe preproliferative diabetic retinopathy, we would separate the DDR grade 0,1,2 images from the DDR grade 3,4 images. We calculate the average and the standard deviations of the means of all DDR grade 0, 1, 2 images against Sort-DR class A. Similarly, we calculate the means and standard deviations of each of the other predictors B,C,D,E,F,G.

Training, validation, testing: The means and standard deviations for the dataset are derived from the training dataset for each AI pairwise comparison model during training. Ten fold cross validations are performed to avoid overfitting.

The Prior probability of needing referral is set to be equal to prevalence of cases that needs referral, as standard procedure. For example, in the DDR dataset, we may wish to separate those with grade DDR0, DDR1, and DDR2 (not needing referrals) from the more severe grades DDR3 and DDR4. The Naïve Bayes method requires us to make an estimate of the prevalence of those images that need referral and those that do not.

Likelihood and likelihood ratios: Given the means and standard deviations, we calculate the likelihoods for each image in the DDR dataset. Combining the likelihood and prior gives the probability that a given image belongs to DDR 012 or alternatively DDR 34. The likelihood ratio tells us how many times a given image is more or less likely to be DDR 34 than DDR 012. In logarithmic form, the likelihood ratio ranges from positive numbers through zero to negative numbers. The likelihood ratio is used to rank the whole DDR image dataset in order of severity.

Cut-offs: Using different cut offs, the sensitivity and specificity, the negative and positive predictive values, the ROC and AUC, and the precision and recall curve are computed. Calibration is performed to determine the “mean, weak and moderate” calibration to describe the behaviour of our model. The frequency, location of errors and their distribution relative to the ranking has been analysed to optimise the negative predictive value of the model.

Amalgamation of models: This was done in one of two ways. The pairwise comparisons for each of the predictors were simply grouped together and the means calculated. Alternatively, the likelihood ratios were multiplied. Both methods assumed independence amongst AI pairwise comparison engines and amongst different predictors.

The basic method of Naive Bayes is only illustrated above. There are further optimisations. The number of predictors does not need to be 7 classes of the Sort-DR. We have 62 steps in the Sort-DR reference database. All or some of the steps could be used to form new classes. Those with less predictive value could be discarded. Different models could be used for different thresholds.

Referring again to FIG. 1, step 56 corresponds to the output, e.g. binary 1 or 0 regarding referral or no referral, together with the certainty value. At step 58, the certainty value is compared to a certainty threshold. The certainty threshold may be set to provide flexibility depending on the competence of the medical professionals involved. If the certainty value is above the certainty threshold, a final recommendation is provided at step 60. If, at step 58, the certainty value is at or below the certainty threshold, the patient's fundus image is sent to a human operator at step 64. After step 64, the final recommendation may be provided at step 60. The final recommendation may be to refer, do not refer, uncertain. For uncertain cases, the decision will be to refer. In other words, there are two outcomes, e.g. to refer or not to refer.

1. A computer-implemented method of determining if a fundus image requires referral for investigation for a disease, the method comprising:
performing a pairwise comparison of the fundus image against each example fundus image in a reference image set using one or more machine learning algorithms to determine a difference in severity of the fundus image compared to each example fundus image in the reference image set; and
flagging the fundus image as requiring referral for investigation for the disease based on results of the pairwise comparisons,
wherein the reference image set includes a plurality of example fundus images, the plurality of example fundus images ranked according to their degree of severity of disease.

2. The computer-implemented method of claim 1 further comprising:
determining a position of the fundus image against the example fundus images within the reference image set; and
comparing the position of the fundus image with a threshold for referral,
wherein the flagging the fundus image as requiring referral comprises flagging the fundus image as requiring referral if the position of the fundus image is above the threshold for referral.

3. The computer-implemented method of claim 1, wherein the one or more machine learning algorithms comprises one or more neural networks.

4. The computer-implemented method of claim 3, wherein the or each neural network is a convolutional neural network.

5. The computer-implemented method of claim 4, wherein the one or more convolutional neural networks is a plurality of convolutional neural networks.

6. The computer-implemented method of claim 5, wherein each of the plurality of convolutional neural networks is trained on a different data set.

7. The computer-implemented method of claim 5, further comprising amalgamating the results of the pairwise comparisons from each convolutional neural network, and wherein the sending the fundus image for referral for investigation for the disease is based on the amalgamated pairwise comparisons.

8. The computer-implemented method of claim 7, wherein the amalgamating of the results of the pairwise comparisons comprises supplying the results of the pairwise comparison as inputs to one or more lasso regression models, the or each lasso regression model having a decision boundary associated with a threshold of disease severity.

9. The computer-implemented method of claim 8, wherein the or each lasso regression model is a plurality of lasso regression models and wherein the threshold of disease severity for each model is different.

10. The computer-implemented method of claim 7, further comprising performing bootstrapping to estimate a certainty value of a probability of needing referral.

11. (canceled)

12. The computer-implemented method of claim 7, wherein the amalgamating the results of the pairwise comparison comprises accumulating results from each convolutional neural network, and comparing the accumulated results to the threshold for referral.

13. The computer-implemented method of claim 7, wherein the amalgamating the results comprises setting a plurality of thresholds of disease severity within the reference image set, and determining a frequency of occurrence of the fundus image above each of the thresholds of disease severity.

14. The computer-implemented method of claim 13, wherein the threshold for referral corresponds to one of the plurality of thresholds of disease severity.

15. The computer-implemented method of claim 7, wherein the amalgamating the results of the pairwise comparison comprises fitting an s-curve to the results of each convolutional neural network; determining a probability of requiring referral based each fitted s-curve; and performing linear discriminant analysis on the determined probabilities.

16. The computer-implemented method of claim 6, further comprising selecting a subset of the plurality of convolutional neural networks using a selecting algorithm.

17. The computer-implemented method of claim 16, wherein the selecting algorithm comprises a lasso regression model, the lasso regression model having a decision boundary associated with the threshold for referral, the selecting comprising applying the results from each convolutional neural network into the lasso regression model, ordering the convolutional neural networks in terms of accuracy at predicting referral, and selecting a predetermined number of the highest ranked convolutional neural networks as the subset.

18. The computer implemented method of claim 1, further comprising:
receiving the reference image set;
performing pairwise comparison of each image of the plurality of images within the reference image set against every other image of the plurality of images within the image set; and
ranking the plurality of example fundus images according to a degree of severity of disease based on the pairwise comparisons.

19. The computer-implemented method of claim 1. wherein the disease is diabetic retinopathy.

20. A computer-implemented method of ranking a plurality of images within a reference image set, the method comprising:
receiving the reference image set;
performing pairwise comparison of each image of the plurality of images within the reference image set against every other image of the plurality of images within the image set; and
ranking the plurality of example fundus images according to a degree of severity of disease based on the pairwise comparisons.

21. A non-transitory computer-readable medium including instructions stored thereon that when executed by one or more processors cause the processor to perform the method of claim 1.
